(19) United States
(12) Patent Application Publication
(10) Pub. No.: US 2021/0100556 A1
(43) Pub. Date: Apr. 8, 2021

(54) INTRODUCER SHEATH WITH ELASTIC PARTITION AND EXPANDABLE OUTER DIAMETER PROFILE

(71) Applicants: SAMIR BIPIN PANCHOLY, CLARKS SUMMIT, PA (US); RAJENDRA GURUDAS SARDESAI, ARCADIA, CA (US)

(72) Inventors: SAMIR BIPIN PANCHOLY, CLARKS SUMMIT, PA (US); RAJENDRA GURUDAS SARDESAI, ARCADIA, CA (US)

(73) Assignee: VASOINNOVATIONS, INC., SOUTH PASADENA, CA (US)

(21) Appl. No.: 17/066,458

(22) Filed: Oct. 8, 2020

Related U.S. Application Data
(60) Provisional application No. 62/912,621, filed on Oct. 8, 2019.

(51) Int. Cl. A61B 17/12

(52) U.S. Cl. CPC A61B 17/12109; A61B 2017/00862; A61B 2017/1205; A61B 17/1204

(57) ABSTRACT

An introducer sheath is provided to facilitate placement of two or more catheters in a patient's vasculature. The introducer sheath includes at least one internal partition thereby providing at least two lumens and the at least one internal partition is made of an elastic material. The internal partition may be made of materials such as polyurethane, silicone, or thermoplastic polymer. The internal partition may also have an expandable fold. The wall of the sheath may also have an expandable fold. An exemplary procedure where an introducer sheath of the present invention may be used is percutaneous coronary intervention on chronic total occlusion in a coronary artery. The introducer sheath of the present invention may also be used to deliver an implant mounted on a catheter.

### CROSS-REFERENCE TO RELATED APPLICATIONS

This application claims benefit of co-pending U.S. Provisional Application No. 62/912621, filed Oct. 8, 2020, the entire content of the which is incorporated herein by reference.

### FIELD OF INVENTION

The invention relates generally to devices, systems, and methods that help deliver catheters or other medical devices to locations within a patient's body. More particularly, the present invention is directed to a sheath for placement of two or more catheters.

### BACKGROUND OF INVENTION

For a variety of endovascular procedures, placement of two catheters is necessary for optimization of procedural success. One of the examples of these type of a circumstance is percutaneous coronary intervention on chronic total occlusion in a coronary artery. In this instance, as the target artery is 100% occluded, while a guidewire is being placed through the occlusion, the distal vessel is not visualized in view of total absence of flow in the segment of the artery beyond the occlusion. Hence the operator has no ability to determine whether the guidewire is in the true lumen of the vessel or has exited the vessel lumen and is extra luminal.

In this instance, the recommended best practice is to place a second catheter in the contralateral artery that provides collaterals to the distal portion of the occluded vessel, and perform an angiogram of the donor vessel providing collaterals, visualize the collaterals and confirm the location of the guide wire that has been advanced through the chronic total occlusion in the target vessel. This requires puncturing a second peripheral artery, femoral, radial, brachial or other artery, and placement of another catheter through that additional punctured access site for the performance of donor vessel angiography to visualize collaterals.

In performing percutaneous coronary intervention on chronic total occlusion of coronary arteries, a technique called retrograde approach is used in some patients. In this technique, two guide catheters are placed, one in the target vessel and the other in the vessel that provides collaterals. The guidewire is then advanced into the vessel that provides collaterals, and is negotiated through the collaterals into the distal segment of the occluded vessel, and the occlusion is crossed in a retrograde fashion. The guidewire is then captured in the guide catheter placed in the target occluded vessel, and once guidewire continuity is established across the occlusion, percutaneous coronary intervention is completed.

Another circumstance where a second catheter and a second access site is needed, is when performing a coronary intervention on a patient with severe left ventricular compromise with high complexity of the target vessel which needs to be intervened upon, in which instance, left ventricular assist device is frequently used to support the left ventricle and provide adequate circulation to other organs during the procedure and afterwards, expecting a further decrease in cardiac function as a result of the procedure.

In these procedures where a second access site is needed, puncturing two arteries to complete the procedure increases the risk of vascular access site complications, especially in these complex substrates of patients, where diffuse multi-segment multisector vascular disease is very common.

### SUMMARY OF THE INVENTION

The devices, systems, and methods for negotiating a patient's vasculature through lumens or vessels are described herein. In particular, the present invention provides improved devices, systems, and methods for procedures including diagnostic, therapeutic, and ablative procedures in arterial and venous systems, as well as for non-vascular lumen and vessel. A catheter system of the present invention comprises a sheath having two or more lumens. In an exemplary embodiment, the multi-lumen sheath of the present invention facilitates the placement of two or more catheters for a variety of endovascular procedures. An example of such procedures is percutaneous coronary intervention on chronic total occlusion in a coronary artery. Embodiments also include a sheath for delivery of an implant mounted on a catheter.

### BRIEF DESCRIPTION OF THE DRAWINGS

FIG. 1 is a schematic depiction of a single lumen introducer sheath and a catheter that is introduced into the sheath.

FIG. 2 illustrates a cross-section of a two-lumen introducer sheath having a lumen divider.

FIG. 3 illustrates a cross-section of a two-lumen introducer sheath having an elastic lumen divider, wherein the elastic divider is pushed to accommodate a large diameter catheter.

FIG. 4 illustrates a cross-section of an introducer sheath comprising multiple lumens.

FIG. 5 illustrates a cross-section of a two-lumen introducer sheath in which the outer wall of the sheath is expandable.

FIG. 6 illustrates a cross-section of a two-lumen introducer sheath in which the wall of the sheath is expanded by unfolding a fold.

FIG. 7 illustrates a cross-section of a two-lumen introducer sheath in which the fold remains open to increase the circumference of the sheath.

FIG. 8a illustrates a cross-section of a two-lumen introducer sheath in which the partition includes a fold.

FIG. 8b illustrates a cross-section of a two-lumen introducer sheath in which the fold in the partition is unfolded to increase the size of the partition.

### DETAILED DESCRIPTION OF THE INVENTION

Embodiments of the present invention are described below with reference to the accompanying drawings. Systems using introducer sheaths according to the present invention provide improved access when placement of two catheters is necessary.

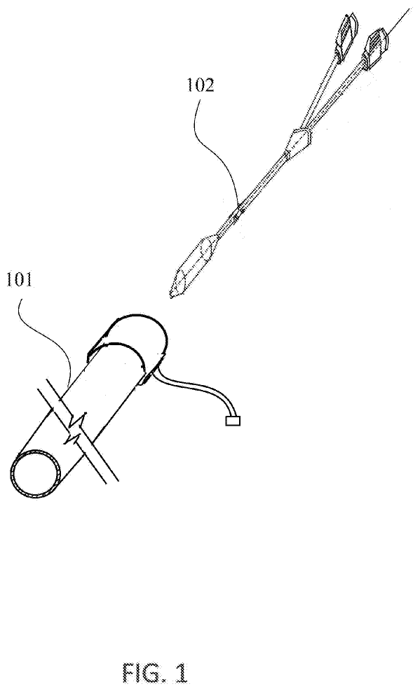

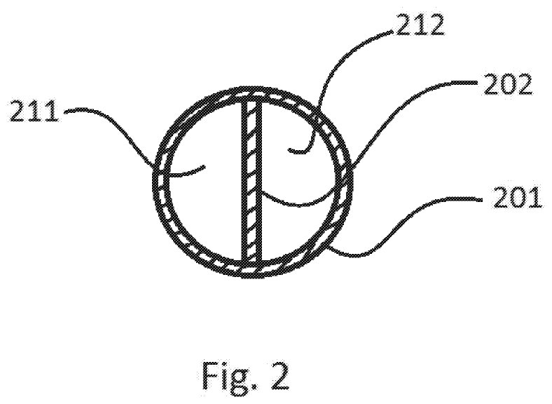

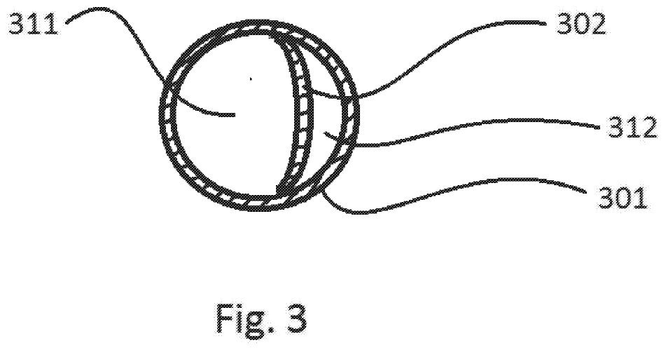

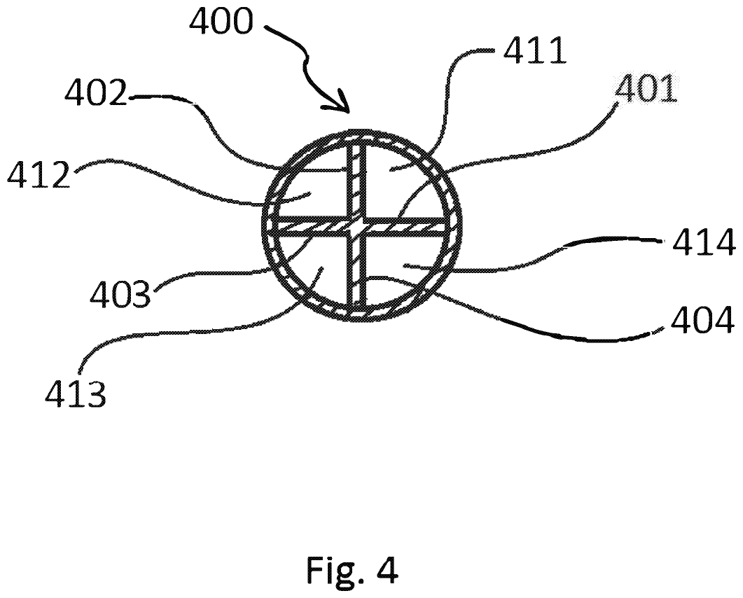

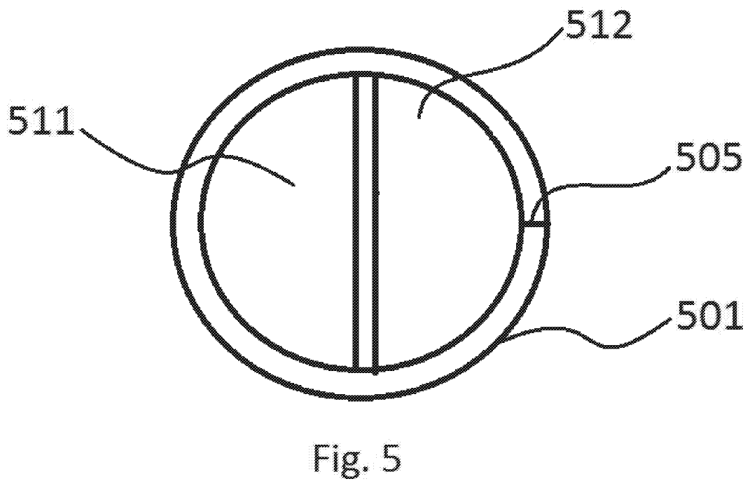

FIG. 1 is a schematic depiction of a single lumen introducer sheath 101 commonly used to introduce a catheter 102 into a patient's vasculature. One embodiment of the present invention (See FIG. 2) comprises a two-lumen introducer sheath 201 with a lumen divider 202 which is elastic, allowing for dynamic variability of the size of each lumen 211 and 212 based on the necessity of the procedure. For example, a larger catheter placed in one lumen 311 in FIG. 3, will push the elastic dividing membrane 302 cannibalizing some of the lumen size of the other lumen 312 in the introducer sheath 301, and vice versa. One embodiment comprises of an introducer sheath with multiple lumens with a wall of at least one lumen being elastic. Another embodiment (see FIG. 4), an introducer sheath 400 comprises multiple lumens with 411, 412, 413, 414 having walls 401, 402, 403, 404, and at least one wall 401 of at least one lumen 411 is elastic. In another embodiment all walls 401, 402, 403, 404 are elastic. In yet another embodiment all walls and the tubular outer body of the introducer catheter is elastic.

Other embodiments with multiple and flexible and elastic partitions provide the ability to insert more than two catheters through a single introducer sheath through a single puncture site.

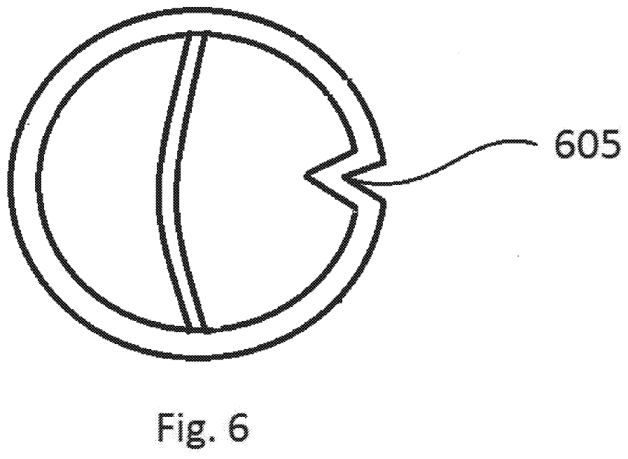

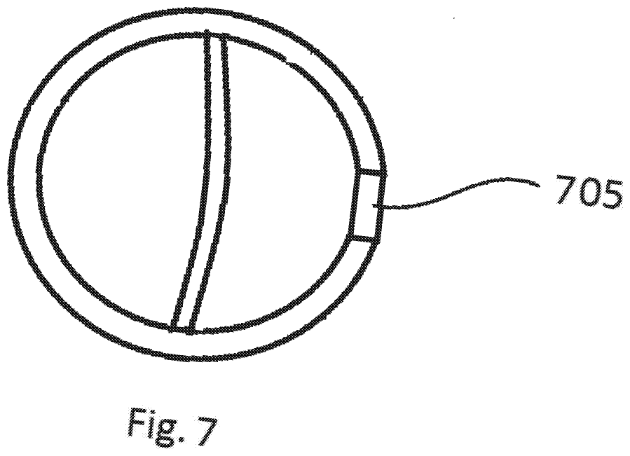

In another embodiment, the introducer sheath is expandable. In one embodiment, the wall of the introducer sheath 501 has a segment 505 which is a fold of the material which in its baseline state is ported upon itself, hence providing a certain diameter of the introducer sheath 501. If multiple catheters need to be introduced in lumen 511, 512, this fold 505 could be unfolded to an opened, unfolded configuration 605 (see FIG. 6), increasing the circumference of the cross-section of the introducer sheath, and hence increasing its ability to accommodate a composite of a larger diameters of multiple catheters. In one embodiment, this fold could be reformed once the additional catheters are removed. In other embodiment (see FIG. 7), the fold 705 may remain in its open confirmation, once unfolded. In another embodiment, multiple folds may be present in the introducer sheath, thereby allowing for expansion of the circumference of the sheath in multiple locations if necessary. In one embodiment where multiple folds are present, one or more of these folds may be selectively deployed. In another embodiment, at least one lumen has an additional partition wall and the tubular wall of the sheath associated with the particular lumen comprises elastic materials and/or expandable walls.

The additional second lumen of the introducer sheath of the present invention may be used for administration of a substance into the vasculature, that may be a medication, contrast material, or other biologically compatible substance, or for monitoring of intraluminal flow or pressure.

The inner partition as also the tubular part of the introducer catheter may be constructed of highly elastic materials with a low tensile set (Thermoplastic elastomer, silicone, polyurethane etc.). In one embodiment, the inner partition is constructed of a relatively elastic material that has enough flexibility to mediate the expansion induced by passage of a large diameter catheter or a large implant, while at the same time having enough material stiffness to urge the inner partition into an approximation of the initial configuration once the large diameter catheter or the large implant has passed. An exemplary material includes NEUSOFT. NEUSOFT is a translucent polyether urethane-based material with good elasticity, abrasion and tear resistance.

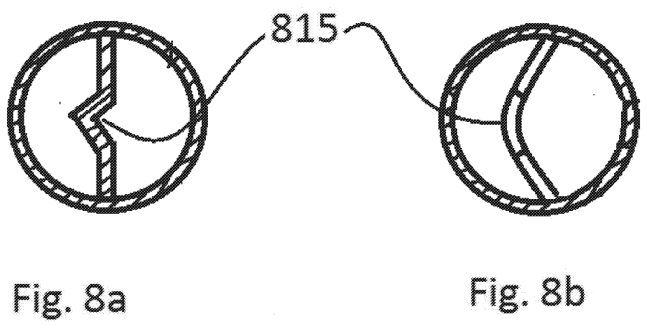

In another embodiment, the wall of the inner partition may be expandable (see FIG. 8a) by including a segment in the partition which is a fold 815 which unfolds to increase the size of the partition (see FIG. 8b) thereby increasing the size of the lumen to accommodate the passage of larger size catheter or device. The fold 815 may be made of the same material as that of the partition or of a different material. In one embodiment the fold 815 is made of a material that is more elastic than the material of the partition.

In one embodiment, an outer diameter of at least a portion of the sheath is from about 3 Fr to about 30 Fr in a non-expanded configuration. In another embodiment, an outer diameter at a distal portion of the sheath is from about 3 Fr to about 30 Fr in a non-expanded configuration. In yet another embodiment, the sheath is tapered and the outer diameter at the proximal end is greater than the outer diameter is at the distal end of the sheath. In another embodiment, the expandable sheath comprises a frame embedded in the tubular wall of the expandable sheath. The frame includes strands extending longitudinally between the proximal end and the distal end, and having an exterior surface and an interior surface that form an interior lumen along the length of the frame. The frame is configured to achieve an expanded state and a contracted state, the expanded state forming an expanded cross-section in the lumen for passing a medical device therethrough. In another embodiment, the frame is located near the outer surface of the introducer sheath and has a smooth coating about the exterior surface and protrusions extending into the wall of the sheath.

In another embodiment, the expandable sheath may include an elastic outer tubular layer and an inner tubular layer. The outer tubular layer defines an initial elastic lumen extending axially therethrough and having an initial diameter. The inner tubular layer has a thick wall portion integrally connected to a thin wall portion—such as by co-extrusion during manufacture. The thick wall portion has a C-shaped cross section with a first longitudinally extending end and a second longitudinally extending end. The thin wall portion extends between the first and second longitudinally extending ends to define an expanded lumen extending axially through the inner tubular layer. The expanded lumen has an expanded diameter larger than the initial diameter of the initial elastic lumen. The inner tubular layer, in a compressed condition, extends through the initial elastic lumen of the elastic outer tubular layer with the elastic outer tubular layer urging the first longitudinally extending end under the second longitudinally extending end of the inner tubular layer. The inner tubular layer in a locally expanded condition has the first and second longitudinally extending ends radially expanded apart, against the urging of the elastic outer tubular layer by passage of a catheter or a device, into a non-overlapping condition with the thin wall portion extending therebetween to form the expanded lumen. The inner tubular layer is configured to be urged by the outer elastic tubular layer into the compressed condition after passage of a catheter or a device through the expanded lumen.

In another embodiment, the outer surface and/or the inner surface of introducer sheath may have a lubricious coating configured to allow smooth sliding. In one embodiment, a radiopaque marker may be located along the length of the sheath. In another embodiment, a radiopaque marker may be fixed to the sheath 101 at its distal end portion, disposed slightly proximal from the tip. In one embodiment, the imaging marker is formed from a radiopaque material (e.g., gold, platinum, tungsten or alloys of these metals or from a silver-palladium alloy, or a platinum-iridium alloy). By so doing, it is possible to confirm the location of the sheath and then to advance the sheath 101 through a patient's vasculature, while monitoring such advancement using radiographic imaging and visualization.

It should be appreciated that several of the above-disclosed and other features and functions, or alternatives or varieties thereof, may be desirably combined into many other different systems or applications. Also, it should be appreciated that various alternatives, derivatives, modifications, variations or improvements thereof or therein may be subsequently made by those skilled in the art, which are also intended to be encompassed by the following claims.

In the description above, for the purposes of explanation, certain requirements and certain details have been included in order to provide an understanding of the embodiments. It will be apparent however, to one skilled in the art, that one or more other embodiments may be practiced without some of the requirements or details. The particular embodiments described are not provided to limit the invention, but merely to illustrate it. The scope of the invention is not to be determined by the specific examples provided above. In other instances, well-known structures, devices, and operations have been shown in block diagram form or without detail in order to avoid obscuring the understanding of the description. Where appropriate, reference numerals or terminal portions of reference numerals have been repeated among the figures to indicate corresponding or analogous elements, which may optionally have similar characteristics.

It should also be appreciated that reference throughout this specification to “one embodiment”, “an embodiment”, “one or more embodiments”, or “different embodiments”, for example, means that a particular feature may be included in the practice of the invention. Similarly, it should be appreciated that in the description various features are sometimes grouped together in a single embodiment, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in fewer than all features of a single disclosed embodiment. In another situation, an inventive aspect may include a combination of embodiments described herein or in a combination of fewer than all aspects described in a combination of embodiments.

1. An introducer sheath comprising:
(a) at least one internal partition thereby providing at least two lumens; and
(b) the at least one internal partition is made of an elastic material.

2. The introducer sheath of claim 1, wherein the partition comprises polyurethane, silicone, or thermoplastic polymer.

3. The introducer sheath of claim 1, wherein an outer diameter of at least a portion of the sheath is from about 3 Fr to about 30 Fr in a non-expanded configuration.

4. The introducer sheath of claim 1, wherein the partition comprises an expandable fold.

5. The introducer sheath of claim 1, wherein the wall of the sheath comprises an expandable fold.
